(19) United States
(12) Patent Application Publication
(10) Pub. No.: US 2007/0042980 A1
(43) Pub. Date: Feb. 22, 2007

(54) Gp131: methods and compositions for treating cancer

(76) Inventors: Ronan C. O'Hagan, Brookline, MA (US); Karuppiah Kannan, Cambridge, MA (US); David Bailey, Brighton, MA (US); Kirk Wright, Waltham, MA (US); Lizabeth Amaral, Metheun, MA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/569,817

(22) PCT Filed: Aug. 27, 2004
(86) PCT No.: PCT/US04/27967

§ 371 Date: Oct. 3, 2006

Related U.S. Application Data
(60) Provisional application No. 60/498,391, filed on Aug. 27, 2003.

(51) Int. Cl. A61K 48/00; C07H 21/02; A61K 39/395; C07K 16/30

(52) U.S. Cl. 514/44; 536/23.1; 424/155.1; 530/388.8

(57) ABSTRACT

The use of molecules relating to the GP131 (ceramide kinase) gene for treating cancer and other hyperproliferative conditions is disclosed. Non-human mammals harboring a genetic modification relating to the GP131 gene, and their use as experimental cancer models, are disclosed.

### FIELD OF THE INVENTION

This invention relates generally to the field of molecular biology. More particularly, this invention relates to genes involved in cancer genesis, maintenance, and progression.

### BACKGROUND OF THE INVENTION

Despite the long history of clinical and research efforts directed towards understanding cancer, surprisingly little is known about the genetic lesions responsible for its genesis, progression, and clinical behavior. For example, in the case of melanoma, although many genes have been implicated in the genesis of this disease, only the INK4a, RAS and BRAF genes have been shown to be true etiologic lesions in a formal genetic sense (Chin et al., Genes Devel. 11:2822-34 (1997); Davies et al., Nature 417:949-54 (2002)). Moreover, advanced malignancy represents the phenotypic endpoint of many successive genetic lesions that affect many oncogene and tumor suppressor gene pathways. Lesions that lead to such a condition may therefore differ from those required to maintain it. Both types of lesions represent rational therapeutic targets in the treatment of cancer.

### SUMMARY OF THE INVENTION

It has been discovered that a gene designated GP131 functionally complements the RAS oncogene in an inducible, spontaneous, in vivo cancer model (mouse). It has also been discovered that interfering RNAs that target GP131 expression inhibit the growth of certain tumor cells in vitro.

Based on these discoveries, the invention provides GP131 antagonists that inhibit GP131 gene expression or GP131 protein activity. Antagonists that inhibit GP131 gene expression include an interfering RNA that inhibits the expression of GP131, a GP131 antisense nucleic acid, and an anti-GP131 ribozyme. Exemplary interfering RNAs of the invention include those that target the sequence of SEQ ID NO: 3, SEQ ID NO: 4, or SEQ ID NO: 5. Antagonists that inhibit GP131 protein activity include blocking antibodies that bind to that portion of the GP131 protein that is exposed on the surface of a GP131-expressing cell. GP131 antagonists of the invention inhibit tumorigenesis, tumor development, tumor maintenance, tumor recurrence, tumor growth, or the growth of tumor cells in vitro.

The invention also provides methods of inducing apoptosis in a cell. The methods include contacting the cell with an effective amount of a GP131 antagonist.

The invention also provides methods of treating a hyperproliferative condition in a mammal, e.g., a human patient. The method includes administering to the mammal an effective amount of a GP131 antagonist. Cancer is an example of such a hyperproliferative condition. Other examples of hyperproliferative conditions are uncontrolled angiogenesis, psoriasis, arteriosclerosis, arthritis and diabetic retinopathy.

In some embodiments, the method of treating a hyperproliferative condition includes administering a second therapeutic agent. The second therapeutic agent can be, for example, an anti-angiogenic agent, anti-metastatic agent, agent that induces hypoxia, agent that induces apoptosis, or an agent that inhibits cell survival signals. Examples of cancer therapeutics include farnesyl transferase inhibitors, tamoxifen, herceptin, taxol, STI571, cisplatin, fluorocil, cytoxan, and ionizing radiation.

The invention also provides a host cell containing a recombinant DNA construct that includes a GP131-encoding sequence operably linked to an expression control sequence, and a genetic mutation that causes the host cell to have a greater likelihood of becoming a cancer cell than a cell not comprising the genetic mutation. Such a mutation can be, e.g., a mutation that deletes or inactivates a tumor suppressor gene, or a mutation that activates an oncogene. Examples of tumor suppressor genes include INK4a, P53, Rb, PTEN, LATS, Apaf1, Caspase 8, APC, DPC4, KLF6, GSTP1, ELAC2/HPC2 and NKX3.1. Examples of oncogenes include K-RAS, H-RAS, N-RAS, EGFR, MDM2, TGF-β, RhoC, AKT family members, myc, β-catenin, PGDF, C-MET, PI3K-CA, CDK4, cyclin B1, cyclin D1, estrogen receptor gene, progesterone receptor gene, HER2 (also known as neu or ErbB2), ErbB1, ErbB3, ErbB4, TGFα, ras-GAP, Shc, Nck, Src, Yes, Fyn, Wnt, and Bcl2.

The invention also provides a genetically modified non-human mammal, e.g., a mouse, at least some of whose cells contain a genome that includes: (a) a recombinant GP131-encoding nucleic acid operably linked to an expression control sequence, and (b) a genetic mutation that causes the mammal to have a greater susceptibility to cancer than a mammal whose cells do not contain the genetic mutation. In preferred embodiments, the genetic mutation involves a tumor suppressor gene and renders the tumor suppressor gene non-functional. The genetically modified nonhuman mammal can be a conventional transgenic mammal, all of whose cells contain the recombinant GP131-encoding nucleic acid operably linked to an expression control sequence, and the genetic mutation that causes the mammal to have increased susceptibility to cancer. Alternatively, the mammal is a chimeric mammal at least some of whose, but not all of whose, somatic cells contain the recombinant GP131-encoding nucleic acid operably linked to an expression control sequence, and the genetic mutation that causes the mammal to have a increased susceptibility to cancer. In such a chimeric mammal, the percentage of somatic cells containing the recombinant GP131-encoding nucleic acid operably linked to an expression control sequence, and a genetic mutation that causes the mammal to have a greater susceptibility to cancer is between 5% and 95%. Preferably it is between 15% and 85%. In some embodiments of the invention, the GP131-encoding nucleic acid is operably linked to a tissue-specific expression system.

The invention also provides a genetically modified nonhuman mammal, wherein the genetic modification reduces or eliminates expression of one or both of the mammal's endogenous GP131 alleles. Such reduction or elimination of GP131 expression can be achieved, for example, when the genetic modification is addition of an RNAi expression construct targeting GP131 gene expression, or when the genetic modification is a knockout of one or both of the GP131 alleles. Such a genetic modification can reduce or eliminate GP131 expression in a tissue-specific manner. In some embodiments of the invention, the genetically modified mammal is chimeric with respect to the genetic modification.

The invention also provides a screening method for identifying a compound useful for treating a hyperproliferative condition such as cancer. The method includes: (a) identifying a biomarker whose level correlates with inhibition of GP131 activity; and (b) detecting a change in the level of the biomarker in the presence of a test compound relative to the level of the biomarker detected in the absence of the test compound.

The invention also provides a screening method for identifying a compound useful in treatment of a hyperproliferative condition such as cancer. The method includes: (a) providing an inhibitor of GP131 expression or activity; (b) identifying a negative control biomarker pattern formed by a plurality of biomarkers in a cancer cell wherein the cell is not contacted with the inhibitor of GP131 expression or activity; (c) identifying a positive control biomarker pattern formed by a plurality of biomarkers in the cancer cell wherein the cancer cell is contacted with the inhibitor of GP131 expression or activity; (d) identifying a test biomarker pattern formed by a plurality of biomarkers in the cancer cell wherein the cancer cell is contacted with a candidate compound but not contracted with the inhibitor of GP131 expression or activity; and (e) comparing the negative control biomarker pattern, positive control biomarker pattern and test biomarker pattern, and detecting a greater similarity between the positive control biomarker pattern and the test biomarker pattern than between the negative control biomarker pattern and the test biomarker pattern.

The invention also provides methods of diagnosing an abnormal hyperproliferative condition, e.g., cancer, in a subject. These methods involve detecting the expression level of a GP131 gene or the activity level of a GP131 protein. An abnormally high level relative to control, e.g., at least about 50%, 100%, 150%, 200%, 250%, or 300% higher, indicates an abnormal hyperproliferative condition.

Unless otherwise defined, all technical and scientific terms used herein have the same meaning as commonly understood by one of ordinary skill in the art to which the invention pertains. In case of conflict, the present specification, including definitions, will control. All publications, patents and other references mentioned herein are incorporated by reference in their entirety.

Throughout this specification and claims, the word “comprise,” or variations such as “comprises” or “comprising” is intended to include the stated integer or group of integers, but not to exclude any other integer or group of integers.

Although methods and materials similar or equivalent to those described herein can be used in the practice or testing of the invention, the preferred methods and materials are described below. The materials, methods and examples are illustrative only, and are not intended to be limiting. Other features and advantages of the invention will be apparent from the detailed description and from the claims.

### BRIEF DESCRIPTION OF THE DRAWINGS

FIG. 1 is a histogram summarizing data on the inhibition of growth of human cancer cell lines DLD-1 (colon), SW620 (colon), LNCAP.FG (prostate) and SK-MEL28 (melanoma) in soft agar by siRNA-mediated knockdown of GP115 expression. Treated cells were transfected with siRNA (SEQ ID NO: 3) targeted against GP131. Negative controls were transfected with siRNA targeted against luciferase. White bars represent GP131 expression level relative to a GP131 expression in negative controls. Black bars represent the number of colonies detectable in the agar after 10 days relative to the number of colonies in the negative controls.

### DETAILED DESCRIPTION OF THE INVENTION

Up-regulation of GP131 contributes to tumorigenesis and tumor maintenance. GP131 was identified as a cancer therapeutic target using by the Mammalian Second Site Supression (“MaSS”) screening system described below and in WO 02/079419.

The GP131 protein, i.e., CERK, is a ceramide kinase. It phosphorylates ceramide at the first position. An exemplary human GP131 protein contains 537 amino acid residues and has the following polypeptide sequence:

(SEQ ID NO: 1; GemBank No. NP_073603)
MGATGAAEPL QSVLWVKQQR CAVSLEPARA LLRWWRSPGP
GAGAPGADAC SVPVSEIIAV EETDVHGKHQ GSGKWQKMEK
PYAFTVHCVK RARRHRWKWA QVTFWCPEEQ LCHLWLQTLR
EMLEKLTSRP KHLLVFINPF GGKGQGKRIY ERKVAPLFTL
ASITTDIIVT EHANQAKETL YEINIDKYDG IVCVGGDGMF
SEVLHGLIGR TQRSAGVDQN HPRAVLVPSS LRIGIIPAGS
TDCVCYSTVG TSDAETSALH IVVGDSLAMD VSSVHHNSTL
LRYSVSLLGY GFYGDIIKDS EKKRWLGLAR YDFSGLKTFL
SHHCYEGTVS FLPAQHTVGS PRDRKPCRAG CFVCRQSKQQ
LEEEQKKALY GLEAAEDVEE WQVVCGKFLA INATNMSCAC
RRSPRGLSPA AHLGDGSSDL ILIRKCSRFN FLRFLIRHTN
QQDQFDFTFV EVYRVKKFQF TSKHMEDEDS DLKEGGKKRF
GHICSSHPSC CCTVSNSSWN CDGEVLHSPA IEVRVHCQLV
RLFARGIEEN PKPDSHS

The sequence of one cDNA encoding this polypeptide is:

(SEQ ID NO: 2; GenBank Accession No. NM_022766)
cccgccgccc gccgcccgcc gcccgggcca ctgcaggggc
cgctaacggt ccggcgcccc tcggcgtccg cgcgccccca
gcctggcgga cgagcccggc ggcggagatg ggggcgacgg
gggcggcgga gccgctgcaa tccgtgctgt gggtgaagca
gcagcgctgc gccgtgagcc tggagcccgc gcgggctctg
ctgcgctggt ggcggagccc ggggcccgga gccggcgccc
ccggcgcgga tgcctgctct gtgcctgtat ctgagatcat
cgccgttgag gaaacagacg ttcacgggaa acatcaaggc
agtggaaaat ggcagaaaat ggaaaagcct tacgctttta
cagttcactg tgtaaagaga gcacgacggc accgctggaa
gtgggcgcag gtgactttct ggtgtccaga ggagcagctg
tgtcacttgt ggctgcagac cctgcgggag atgctggaga
agctgacgtc cagaccaaag catttactgg tatttatcaa
cccgtttgga ggaaaaggac aaggcaagcg gatatatgaa
agaaaagtgg caccactgtt caccttagcc tccatcacca
ctgacatcat cgttactgaa catgctaatc aggccaagga
gactctgtat gagattaaca tagacaaata cgacggcatc
gtctgtgtcg gcggagatgg tatgttcagc gaggtgctgc
acggtctgat tgggaggacg cagaggagcg ccggggtcga
ccagaaccac ccccgggctg tgctggtccc cagtagcctc
cggattggaa tcattcccgc agggtcaacg gactgcgtgt
gttactccac cgtgggcacc agcgacgcag aaacctcggc
gctgcatatc gttgttgggg actcgctggc catggatgtg
tcctcagtcc accacaacag cacactcctt cgctactccg
tgtccctgct gggctacggc ttctacgggg acatcatcaa
ggacagtgag aagaaacggt ggttgggtct tgccagatac
gacttttcag gtttaaagac cttcctctcc caccactgct
atgaagggac agtgtccttc ctccctgcac aacacacggt
gggatctcca agggatagga agccctgccg ggcaggatgc
tttgtttgca ggcaaagcaa gcagcagctg gaggaggagc
agaagaaagc actgtatggt ttggaagctg cggaggacgt
ggaggagtgg caagtcgtct gtgggaagtt tctggccatc
aatgccacaa acatgtcctg tgcttgtcgc cggagcccca
ggggcctctc cccggctgcc cacttgggag acgggtcttc
tgacctcatc ctcatccgga aatgctccag gttcaatttt
ctgagatttc tcatcaggca caccaaccag caggaccagt
ttgacttcac ttttgttgaa gtttatcgcg tcaagaaatt
ccagtttacg tcgaagcaca tggaggatga qgacagcgac
ctcaaggagg gggggaagaa gcgctttggg cacatttgca
gcagccaccc ctcctgctgc tgcaccgtct ccaacagctc
ctggaactgc gacggggagg tcctgcacag ccctgccatc
gaggtcagag tccactgcca gctggttcga ctctttgcac
gaggaattga agagaatccg aagccagact cacacagctg
agaagccggc gtcctgctca caaactggga aagtgtgaaa
actatttaag ataattatta cagaccaatt atgttgatat
atacatttaa atgtagaaat ttatttttga tagttaaatc
ttgattttag aagaaaaccc ttttgtcaac aattttgtgt
acatatttgg cattttcagt tctgtacgca tctgcgggtt
gcagcccacg ccgcttactc tcagcggatg cagctgctca
cttgggggca ctggcctctt aggttttaac gatgtcaaca
gtgtagttta gaaaatggcc cgttagtggc tctattgcaa
taatgttagg gacattatat gatttccacg caggtcacac
catctgggcc tgaggtagca gtgggtcact ttgatccact
ttgcaggact tattctgtaa cggtttgtgg ccaagttttg
ggaagtggtt gattctcttt gccttcattt caccttcctc
ttcgtttacg gttaggacat cgctgcttga tccttacaat
actgtgcaac tgcaatgcaa cgtggccctg cttcaggtga
tccgcgggag gggcctccac gccagcgccg ggaaggctgc
tggggcctcc acacctgcct catcacggcg gcgaggctac
gacaatccgg ctgggagcat gaccttggcg tctgttctgg
gagcacggat gataagctct ggaagctggc agtgtgtaaa
gcactggcaa gtttgttact gttaaaatgt caaataccaa
tgctttatat cgacgcgaag tgcttaacac agccgggctt
gggggcagtc aggaggaagc tggccatccg tggaggaggg
gccggtcctg gactcccgca ggactcctct gaggcagggc
ctgaagtctg tacacgtggt ccagatttgt ccttgtcttt
tcttcacact gagttctcta tatttattga acatcttgtc
cttttaagcc agagtagtgt aaactgcgtc tcggatgtct
gtcttttgcc tcgaagccac gatggatcgc tggtttcctc
tgcagcgcga gggctccggc gaccagagga ttcttcccgg
aaggcattcc tgccgcgctc cccggggcac ccctcaattg
tgtactacgt ccttgtttag tgtgtatccg tgcccacgta
gatgatgtct gtaacgtagt tttgtttgaa atatgagaat
atgcggctta aactttgatc tgtaaggagc ggggccgtgg
ccgtttggag cacgctgtag acaccgttcc tcatgctgcc
gggtgggttt tgcagaagct cccttagtga tttcatgttt
aacaggcagc atccattttc agaatttcct ggcattgatt
tatattttga agcatacagg aaacttctcg tttcctcgtt
tagccccacc cagatcaggt gaaagggcag ctttaatggt
ggtttttatg gaccacatta tcagagagca ctgtgcaagc
caaatggttc aataatgaat gaaaattctg ggtgtaaaga
gtaaatatgc cctggctctt tctaccaatg tttgctcctg
gttggaaaga aaccaaagat ttaagacggg ctgctcttcc
agactggctg tgcctgcctg tgcccagcaa cctgtgcagc
cggcagtgtg cctggtgtca cgccaggagg ctgtggctgc
tgtgggccct ctggaattgt gctcctcaca aagtttcccc
aaaaggttct tctaagcctt tattgtccct ggtaaatgtt
tcccggctgg gcgcggtggc tcacgcctgt aatcccagca
ctttgggagg ccgaggcggg tggatcacct aaggtcagga
gtttgagatc agcctgccca acatggtgaa acctcgtctc
tactaaaaat acacaactta gccagtcttg ttggcgcacg
cctgtaatct cagctactag ggatgctgag gcaggagaat
cgcttgaacc caagaggtgg aggttgcggt gagccaagat
tgcgccactg cactccagcc tgggcaaaca gagggagact
ccatcgcccc ccccaacaaa aaaaaaagtt tcccatacac
tggcctgccc caaaacccac taacaatttt agcaaaacag
tccaggccaa agaggaagca tttcatgttc aataagaaac
ccagccattc cgcatggctg gttcctgagt ggctctggtg
atactctcca gccacctgct gacattcaga atctcagacc
tcgggactgc tgttgcggta ccgtgtgtct gacacctgcc
agcagccctt tgctatctgc gcgcaggatg ggggtgactg
cccagacatt cccgctagat aggctctgat ttccggggca
gcctttcaga tgcggcagac atacaacacc tgtactttag
agttttaagg gaaaaaaaat cagaagtgct ggttagatag
taaaaactta ggataactta gaaaggctag ttttagcttc
ctttgtggct ccctggtgca aaacaattag cagttatgca
atggacctga ttctagttta ttctaattaa gaagtgaggc
cgagtttgac ttcgttcctg aatacaatct tgagtaactg
ggaaagtctg agtgaaagga tggcctcatt ctctttctaa
tcttgctggt ttcaagatta gaaaatggca ttatttgatc
tgaaatgttt gagaagacac gaataaagtt acttgggcag
aaaaaaaaaa aaaaaaa

The open reading frame of the above sequence is nucleotides 108-1721. Other human GP131 polypeptide sequences include GenBank accession numbers NP_{—}872602. Other human GP131 coding sequences include Genbank accession numbers AL137600.1, AK025083.1, AK026892.1, NM_{—}022766.3, AB051433.1, BC004278.1, BC008382.1, AJ457828.1, AB079066.1, and AL832794.1. GP131 orthologs in other animal species have also been identified. They include GenBank accession numbers NM_{—}145475 (M. musculus), and NM_{—}059576 (C. elegans).

The GP131 gene is expressed in a variety of tissues, including colon, kidney, lung, heart, myeloid cells, stomach, cervix, uterus, endometrium, B-cells, liver, spleen, melanocyte, marrow, placenta, blood, brain, testis, leukocyte, islets of Langerhans, ascites, osteoarthritic cartilage and breast. The expression of this gene is increased in a broad spectrum of cancer tissue types (e.g., retinoblastoma, hypernephroma, ovarian tumor, squamous cell carcinomas, melanotic melanoma, chronic lymphotic leukemia, head and neck cancer, leiomyosarcoma, anaplastic oligodendroglioma and metastatic chondrosarcoma) and in cancer cell lines (e.g., adenocarcinoma cell line, high MDR cell line, epidermoid carcinoma cell line, cervical carcinoma cell line, lymphoma cell line, hepatocellular carcinoma cell line, ductal carcinoma cell line, human chondrosarcoma cell line, HCT-116, DLD-1, SK-MEL28, A549, MiaPaCa2, LNCaP.FGC, 22Rv1, DU145, MCF-7, PC-3, SW620, HCT-15, and KATO III).

The GP131 gene was found to be involved in development (including maintenance, progression, angiogenesis, and/or metastasis) of cancers, e.g., cancers found in skin (e.g., melanoma), lung, prostate, breast, colorectal, liver, pancreatic, brain, testicular, ovarian, uterine, cervical, kidney, thyroid, bladder, esophageal, and hematological tissues. The cancer-related functions of this gene can be confirmed by, e.g., (1) its overexpression in more than about 10% of human cancers; (2) the inhibition of human cancer cell proliferation and growth in soft agar by RNA interference (“RNAi”) of this gene; (3) the ability of this gene to enhance transformation of MEFs by myc and ras-V12; (4) the ability of this gene to enhance the growth of mouse tumor cell lines in soft agar; and/or (5) the prevention by inhibiting this gene of maintenance or formation of tumors arising de novo in a mouse, or tumors derived from human cancer cell lines.

I. GP131-Related Nucleic Acids

The nucleic acid sequences specifically provided herein are sequences of deoxyribonucleotides. However, the given sequences are to be interpreted as would be appropriate to the polynucleotide composition. For example, if the isolated nucleic acid is RNA, the given sequence intends ribonucleotides, with uridine substituted for thymidine. In some embodiments, differences from naturally occurring nucleic acids, e.g., non-native bases, altered internucleoside linkages, and post-synthesis modification, can be present throughout the length of the GP131 nucleic acid or can be usefully localized to discrete portions thereof. For example, a chimeric nucleic acid can be synthesized with discrete DNA and RNA domains and demonstrated utility for targeted gene repair. See, e.g., U.S. Pat. Nos. 5,760,012 and 5,731,181.

Polymorphisms such as single nucleotide polymorphisms (SNPs) occur frequently in eukaryotic genomes. Additionally, small deletions and insertions, rather than SNPs, are not uncommon in the general population, and often do not alter the function of the protein. Accordingly, this invention provides not only isolated nucleic acids identical in sequence to those described with particularity herein, but also isolated nucleic acids that are allelic variants of those particularly described nucleic acid sequences. In some embodiments, such sequence variations result from human intervention, e.g., by random or directed mutagenesis.

A. Nucleic Acids Encoding GP131 Protein or Portions Thereof

The invention provides isolated nucleic acid molecules that encode the entirety or part (e.g., at least five, seven, or nine contiguous amino acid residues) of the GPC protein, including allelic variants of this protein. As is well known, the genetic code is degenerate and codon choice for optimal expression varies from species to species. Thus, the coding sequences of this invention include degenerate variants of the sequences described herein with particularity. Thus, the isolated polynucleotide comprises a nucleotide sequence encoding SEQ ID NO: 1.

These nucleic acids can be used, for example, to express the GP131 protein or specific portions of the protein, either alone or as elements of a fusion protein, e.g., to express epitopic or immunogenic fragments of the GP131 protein. For example, such nucleic acids are used to produce non-human mammals of the invention. These nucleic acids also can be used also as probes to hybridize to GP131 nucleic acids and related nucleic acid sequences.

This invention also relates to nucleic acids comprising sequences coding for polypeptides containing conservative amino acid substitutions or moderately conservative amino acid substitutions from those polypeptides described with particularity herein. These amino acid substitutions can be due to, e.g., allelic variations, naturally occurring mutations, or manmade mutations.

B. Cross-Hybridizing Nucleic Acids

This invention also relates to isolated polynucleotides that hybridize to one or more of the above-described GP131 nucleic acids. These cross-hybridizing nucleic acids can be used, e.g., as hybridization probes, primers, and/or for expression of proteins that are related to GP131 as isoforms and homologs, e.g., paralogs, and orthologs. In some such embodiments, the invention relates to an isolated nucleic acid comprising a sequence that hybridizes under high stringency conditions to a probe comprising a fragment of SEQ ID NO: 2 having at least 15, 16, 18, 20, 24, or 25 nucleotides. As used herein, “high stringency conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The hybridizing portion of a reference nucleic acid (i.e., target nucleic acid) is typically at least 15 nucleotides in length, and often at least 17, 20, 25, 30, 35, 40 or 50 nucleotides in length. Cross-hybridizing nucleic acids that hybridize to a larger portion of the reference nucleic acid—for example, to a portion of at least 50, 100, 150, 200, 250, 300, 350, 400, 450, 500 or more nucleotides, up to and including the entire length of the reference nucleic acid, are also useful.

C. Nucleic Acid Fragments

Fragments of the above-described nucleic acids also relates to in this invention. They can be used as region-specific probes, as amplification primers, regulatory sequences to direct expression of a gene, and/or to direct expression of a GP131 polypeptide fragment, e.g., immunogenic fragment.

The nucleic acid probes may comprise a detectable label, e.g., a radioisotope, a fluorescent compound, an enzyme, or an enzyme co-factor. Such probes can be used as a part of a diagnostic kit for identifying cells or tissues that (i) incorrectly express a GP131 protein, e.g., aberrant splicing, or abnormal mRNA levels, or (ii) harbor a mutation in the GP131 gene, such as a deletion, an insertion, or a point mutation. Such diagnostic kits preferably include labeled reagents and instructional inserts for their use.

The nucleic acid primers can be used in PCR, primer extension and the like. They can be, e.g., at least 6 nucleotides (e.g., at least 7, 8, 9, or 10) in length. The primers can hybridize to an exon sequence of a GP131 gene, e.g., for amplification of a GP131 mRNA or cDNA. Alternatively, the primers can hybridize to an intron sequence or an upstream or downstream regulatory sequence of a GP131 gene, to utilize non-transcribed, e.g., regulatory portions of the genomic structure of a GP131 gene. The nucleic acid primers also can be used, e.g., to prime single base extension (SBE) for SNP detection (see, e.g., U.S. Pat. No. 6,004,744). Isothermal amplification approaches, such as rolling circle amplification, are also now well-described. See, e.g., Schweitzer et al., Curr. Opin. Biotechnol. 12(1):21-7 (2001); U.S. Pat. Nos. 5,854,033 and 5,714,320 and international patent publications WO 97/19193 and WO 00/15779. Rolling circle amplification can be combined with other techniques to facilitate SNP detection. See, e.g., Lizardi et al., Nature Genet. 19(3):225-32 (1998).

Nucleic acid fragments that encode 5 or more contiguous amino acids, e.g., fragments of 15, 18, 21, 24, or 27 nucleotides or more, are useful in directing the synthesis of peptides that have utility in mapping the epitopes of the GP131 protein. See, e.g., Geysen et al., Proc. Natl. Acad. Sci. USA 81:3998-4002 (1984); and U.S. Pat. Nos. 4,708,871 and 5,595,915. Such nucleic acid fragments are also useful in directing the synthesis of peptides that have utility as immunogens. See, e.g., Lerner, “Tapping the immunological repertoire to produce antibodies of predetermined specificity,” Nature 299:592-596 (1982); Shinnick et al., Annu. Rev. Microbiol. 37:425-46 (1983); Sutcliffe et al., Science 219:660-6 (1983).

Of course, larger fragments containing at least 25, 30, 35, 40, 45, 50, 100, 150, 200, 250, 300, 350, 400, 450, 500 or more nucleotides are also useful and sometimes preferred.

D. Single Exon Probes

The invention further relates to single exon probes having portions of no more than one exon of the GP131 gene. Such single exon probes have particular utility in identifying and characterizing splice variants. In particular, these probes are useful for identifying and discriminating the expression of distinct isoforms of GP131.

E. Antisense Reagents

1. Antisense Nucleic Acids

Some embodiments of the invention relate to isolated nucleic acids that are antisense polynucleotides that specifically hybridize to GP131 sense polynucleotides. The antisense nucleic acid molecule can be complementary to the entire coding or non-coding region of GP131, but more often is antisense to only a portion of the coding or non-coding region of GP131 mRNA. For example, the antisense oligonucleotide can be complementary to the region surrounding the translation start site of GP131 mRNA. An antisense oligonucleotide can be, for example, about 5, 10, 15, 20, 25, 30, 35, 40, 45 or 50 nucleotides in length.

The antisense nucleic acids of this invention may, for example, form a stable duplex with its target sequence, or, in the case of an antisense nucleic acid molecule that binds to DNA duplexes, through specific interactions in the major groove of the double helix. In yet other embodiments, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. Nucl. Acids Res 15: 6625-6641 (1987)).

An antisense target sequence is a nucleotide sequence specific to GP131, and can be designed through use of a publicly available sequence database, and/or through use of commercially available sequence comparison programs. Antisense nucleic acids of the invention can then be constructed using procedures known in the art. For example, an antisense nucleic acid (e.g., an antisense oligonucleotide) can be produced biologically using an expression vector into which a nucleic acid has been inserted in an antisense orientation, i.e., RNA transcribed from the inserted nucleic acid will be of an antisense orientation to a target nucleic acid of interest.

Alternatively, the antisense nucleic acid can be chemically synthesized using naturally occurring nucleotides or variously modified nucleotides designed to increase the biological stability of the molecule or to increase the physical stability of the duplex formed between the antisense and sense nucleic acids. For example, phosphorothioate derivatives and acridine substituted nucleotides can be used. Phosphorothioate and methylphosphonate antisense oligonucleotides are useful in practicing the invention. The antisense nucleic acid molecule can also comprise a 2′-O-methylribonucleotide (Inoue et al., Nucl. Acids Res 15: 6131-6148 (1987)) or a chimeric RNA-DNA analogue (Inoue et al. FEBS Lett 215: 327-330 (1987)). The antisense oligonucleotides may be further modified by adding poly-L-lysine, transferrin, polylysine, or cholesterol moieties at their 5′ end.

According to the invention, antisense molecules can be administered to a subject, e.g., a mouse or human) or generated in situ via an expression vector, such that they bind to cellular RNA and/or genomic DNA encoding a GP131 protein thereby inhibiting GP131 expression by inhibiting transcription and/or translation. Suppression of GP131 expression at either the transcriptional or translational level is useful to treat certain cancer conditions in patients or to generate cellular or animal models for cancer characterized by aberrant GP131 expression. An antisense molecule can be administered by direct injection at a tissue site of a subject. Alternatively, an antisense molecule can be designed to target selected cells, e.g., cancer cells overexpressing GP131, and then administered systemically.

2. Ribozymes and Catalytic Nucleic Acids

In some embodiments, an antisense nucleic acid of the invention is part of a GP131-specific ribozyme (or, as modified, a “nucleozyme”). Ribozymes are catalytic RNA molecules with ribonuclease activity capable of cleaving a single-stranded nucleic acid, such as an mRNA, to which they have a complementary region. Thus, ribozymes such as hammerhead, hairpin, and Group I intron ribozymes can cleave GP131 mRNA transcripts catalytically, thereby inhibiting translation of GP131 mRNA. A ribozyme having specificity for a GP131-encoding nucleic acid can be designed based upon the nucleotide sequence of a GP131 polynucleotide disclosed herein (SEQ ID NO: 2). See, e.g., U.S. Pat. Nos. 5,116,742; 5,334,711; 5,652,094; and 6,204,027. For example, a derivative of a Tetrahymena L-19 IVS RNA can be constructed in which the nucleotide sequence of the active site is complementary to the nucleotide sequence to be cleaved in a GP131-encoding mRNA. See, e.g., Cech et al. U.S. Pat. Nos. 4,987,071 and 5,116,742. Alternatively, GP131 mRNA can be used to select a catalytic RNA having a specific ribonuclease activity from a pool of RNA molecules. See, e.g., Bartel et al., Science 261:1411-1418 (1993).

In some embodiments, the ribozymes and other antisense reagents of this invention include appended groups such as peptides. This is useful for targeting host cell receptors, facilitating transport across the cell membrane (Letsinger et al., Proc. Natl. Acad. Sci. USA 86:6553-6556 (1989); Lemaitre et al., Proc. Natl. Acad. Sci. USA 84:648-652 (1987); WO 88/09810), or facilitating transport across the blood-brain barrier (WO 89/10134).

Expression of the GP131 gene can be inhibited by targeting nucleotide sequences complementary to the regulatory region of the GP131, e.g., the GP131 promoter and/or enhancers, to form triple helical structures that prevent transcription of the GP131 gene in target cells. See generally, Helene, Anticancer Drug Des. 6: 569-84 (1991); Helene et al. Ann. N.Y. Acad. Sci. 660:27-36 (1992); and Maher Bioassays 14: 807-15 (1992).

3. Peptide Nucleic Acids (PNA)

Some preferred oligonucleotide mimetics, especially those useful for in vivo administration, are peptide nucleic acids (PNA). See, e.g., Hyrup et al. Bioorg. Med. Chem. Lett. 4:5-23 (1996). In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza-nitrogen atoms of the amide portion of the backbone, typically by methylene carbonyl linkages. The synthesis of PNA oligomers can be performed using conventional solid phase peptide synthesis as described in Hyrup et al., supra; and Perry-O'Keefe et al., Proc. Natl. Acad. Sci. USA 93:14670-675 (1996).

GP131-based PNAs can be used in therapeutic and diagnostic applications. For example, PNAs can be used as antisense or antigene agents for sequence-specific modulation of gene expression by, e.g., inducing transcription or translation arrest or inhibiting replication. GP131-based PNAs also can be used in the analysis of single base pair mutations in a gene. This can be done by PNA directed PCR clamping; as artificial restriction enzymes when used in combination with other enzymes, e.g., S1 nucleases, or as probes or primers for DNA sequence and hybridization (Hyrup et al., supra; Perry-O'Keefe, supra).

In other embodiments, PNAs of GP131 are modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of GP131 can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup et al., supra). The synthesis of PNA-DNA chimeras can be performed as described in Hyrup, supra and Finn et al., Nucl. Acids Res. 24:3357-63 (1996).

4. RNA Interference

The invention provides RNA interference (RNAi) for use in silencing the expression of the GP131 gene. RNAi is a sequence-specific posttranscriptional gene silencing mechanism triggered by double-stranded RNA (dsRNA) and causes degradation of mRNAs homologous in sequence to the dsRNA. The mediators of the degradation are 21- to 23-nucleotide small interfering RNAs (siRNAs) generated by ribonuclease III cleavage from the longer dsRNAs. Molecules of siRNA typically have 2- to 3-nucleotide 3′ overhanging ends resembling the RNAse III processing products of long dsRNAs that normally initiate RNAi. When introduced into a cell, they assemble with an endonuclease complex (RNA-induced silencing complex), which then guides cleavage of the targeted mRNA. This results in a phenotype with suppressed expression of the protein encoded by the targeted mRNA. The small size of siRNAs avoids activation of the dsRNA-inducible interferon system in mammalian cells. This helps avoid the nonspecific phenotypes normally produced by dsRNA larger than 30 base pairs in somatic cells. For review, see, e.g., Elbashir et al., Methods 26:199-213 (2002); McManus and Sharp, Nature Reviews 3:737-747 (2002); Hannon, Nature 418:244-251 (2002); Tuschl, Nature Biotechnology 20:446-448 (2002); and Tuschl U.S. Application US2002/0086356 A1.

SiRNA oligonucleotides can be designed with commercially available software programs, e.g., the OligoEngine siRNA design tool available at http://www.oligoengine.com. Preferred siRNAs of this invention range about 19-29 basepairs in length for the double-stranded portion. In some embodiments, the siRNAs are hairpin RNAs having an about 19-29 bp stem and an about 4-34 nucleotide loop. In some embodiments, siRNAs are highly specific for a GP131 target region and may comprise any 19-29 bp fragment of a GP131 mRNA that has at least 1 (e.g., at least 2 or 3) bp mismatch with a non-GP131-related sequence. In some embodiments, the GP131 siRNAs do not bind to RNAs having more than 3 mismatches. The target sequences of exemplary siRNAs for GP131 are:

| | |
|---|---|
| 5′- gatcatcgccgttgaggaa -3′; | (SEQ ID NO: 3) |
| 5′- caaggcaagcggatatatg -3′; | (SEQ ID NO: 4) |
| and | |
| 5′- ctgacatcatcgttactga -3′. | (SEQ ID NO: 5) |

These three sequences correspond to nucleotides 275-293, 540-558 and 601-619 of SEQ ID NO: 2, respectively. SiRNAs that target an RNA region having 10 or more nucleotide overlaps with an aforementioned exemplary target region are also useful.

Intracellular transcription of siRNAs can be achieved by cloning the siRNA templates into RNA polymerase III (Pol III) transcription units, which normally encode the small nuclear RNA U6 or the human RNAse P RNA H1. Two approaches can be used for expressing siRNA: (1) sense and antisense strands constituting the siRNA duplex are transcribed by individual promoters; or (2) siRNAs are expressed as fold-back stem-loop structures that give rise to siRNAs after intracellular processing. Inducible promoters can also be used to drive the expression of the siRNA.

By way of example, to target specific regions of a GP131 mRNA, the following oligonucleotides can be used with a primer specific to the U6 small RNA promoter to form double-stranded DNA in a polymerase chain reaction, using a vector containing this U6 promoter as a template. The PCR product can then be ligated into a vector. Expression of the insert leads to expression of a short hairpin RNA. The hairpin structure displays inhibitory effects on GP131 expression.

1) GP 131 target nucleotides 1-29 (in boldface)

(SEQ ID NO: 6)

ggaattcaaaaaggcccgggcagcgggcggcggacgacgagccaacccgc
cgcccgccgcccgccgcccgggcctagtatatgtgctgccgaagc
2) GP131 target nucleotides 4225-53 (in boldface)

(SEQ ID NO: 7)

ggaattcaaaaagaaccaggtccattgcataaccgccaactccaaaatta
gcagttatgcaatggacctgattctagtatatgtgctgccgaagc
3) GP131 target nucleotides 1561-89 (in boldface)

(SEQ ID NO: 8)

ggaattcaaaaagacggtgcaacagcaagagggatggctacccaagcagc
cacccctcctgctgctgcaccgtctagtatatgtgctgccgaagc
4) GP131 target nucleotides 615-43 (in boldface)

(SEQ ID NO: 9)

ggaattcaaaaagtctccttgacctgactagcacgttcaatccaaactga
acatgctaatcaggccaaggagactagtatatgtgctgccgaagc

An siRNA oligonucleotide or its coding sequence can be delivered into a target cell via a variety of methods, including but not limited to, liposome fusion (transposomes), routine nucleic acid transfection methods such as electroporation, calcium phosphate precipitation, and microinjection, and infection by viral vectors.

F. Exemplary Uses of GP131 Nucleic Acids

1. Characterization of Genetic Mutations

The above-described isolated nucleic acids can be used as hybridization probes to characterize GP131 nucleic acids in both genomic and transcript-derived nucleic acid samples. For example, the probes can be used to detect gross alterations in the GP131 genomic locus, such as deletions, insertions, translocations, and duplications of the GP131 genomic locus. Methods of detection include fluorescence in situ hybridization (FISH) to chromosome spreads, comparative genomic hybridization (CGH), array CGH (e.g., on microarrays containing GP131-coding sequences or BAC comprising GP131-coding sequences), and spectral karyotyping (SKY). See, e.g., Andreeff et al. (eds.), Introduction to Fluorescence In Situ Hybridization: Principles and Clinical Applications, John Wiley & Sons (1999) (ISBN: 0471013455). The probes also can be used to assess smaller genomic alterations using, e.g., Southern blot detection of restriction fragment length polymorphisms. The nucleic acid probes can be also used to isolate genomic clones that include the nucleic acids of the present invention, which thereafter can be restriction mapped and sequenced to identify deletions, insertions, amplifications, translocations, and substitutions (e.g., SNPs) at the sequence level. The nucleic acid probes can also be used to isolate GP131 nucleic acids from cDNA libraries, permitting sequence level characterization of GP131 RNA messages, including identification of deletions, insertions, truncations—including deletions, insertions, and truncations of exons in alternatively spliced forms—and single nucleotide polymorphisms. Some of the nucleic acids also can be used as amplification primers for real time PCR to detect the above-described genomic alterations. Such genomic alterations of the GP131 gene often play a role in tumor genesis, maintenance and development, and thus can be used as markers for diagnosis and prognosis of GP131-mediated cancers.

2. Quantification of Expression Levels

The nucleic acid probes can be used to measure the representation of GP131 clones in a cDNA library, used as primers for quantitative real time PCR, or otherwise used to measure expression level of the GP131 gene. Measurement of GP131 expression has particular utility in diagnostic assays for cancer-related conditions associated with abnormal GP131 expression. Moreover, differences in the expression levels of the gene before and after a cancer event (e.g., cancer genesis, maintenance, regression, and metastasis) are useful in determining the effect of a candidate cancer drug, identifying cancer types, designing diagnostics and prognostics, and predicting likely outcome of a cancer therapy.

3. Genetic Alterations

The nucleic acids also can be used to introduce mutations (e.g., null mutations, dominant negative mutations, dominant acting mutations) into a GP131 locus of an animal via homologous recombination. Such animals (e.g., knock out mice) are useful in delineating the role of GP131 in tumor genesis and development and in facilitating cancer drug development.

Where the genomic region includes transcription regulatory elements, homologous recombination can be used to replace the endogenous regulatory elements with heterologous regulatory elements, i.e., elements not natively associated with the gene in the same manner. This can alter the expression of GP131, both for production of GP131 protein, and for gene therapy. See, e.g., U.S. Pat. Nos. 5,981,214, 6,048,524; and 5,272,071.

Fragments of the above-described polynucleotides smaller than those typically used for homologous recombination can also be used for targeted gene correction or alteration, possibly by cellular mechanisms different from those engaged during homologous recombination. See, e.g., U.S. Pat. Nos. 5,945,339, 5,888,983, 5,871,984, 5,795,972, 5,780,296, 5,760,012, 5,756,325, 5,731,181; and Culver et al., Nature Biotechnol. 17(10):989-93 (1999); Gamper et al., Nucl. Acids Res. 28(21):4332-9 (2000).

II. Vectors and Host Cells

A. General Consideration

This invention relates to nucleic acid constructs containing one or more of the isolated nucleic acid molecules encoding all or part of GP131. The vectors can be used to propagate the new nucleic acid molecules in host cells, to shuttle the molecules between host cells derived from disparate organisms, to insert the molecules into host genomes, to express sense or antisense RNA transcripts or interfering RNAs, and/or to express GP131 polypeptides. Typically, the vectors are derived from virus, plasmid, prokaryotic or eukaryotic chromosomal elements, or some combination thereof, and may optionally include at least one origin of replication, at least one site for insertion of heterologous nucleic acid, and at least one selectable marker.

This invention relates to host cells, which can be either prokaryotic (bacteria) or eukaryotic (e.g., yeast, insect, plant and animal cells). A host cell strain may be chosen for its ability to process the expressed protein in the desired fashion. Such post-translational modifications of the polypeptide include, but are not limited to, acetylation, carboxylation, glycosylation, phosphorylation, hydroxylation, sulfation, lipidation, and acylation. Some embodiments of the invention may involve GP131 proteins with such post-translational modifications.

Exemplary prokaryotic host cells are E. coli, Caulobacter crescentus, Streptomyces species, and Salmonella typhimurium cells. Vectors useable in these cells include, without limitation, those related to pBR322 and the pUC plasmids.

Exemplary yeast host cells are Saccharomyces cerevisiae, Schizosaccharomyces pombe, Pichia pastoris, and Pichia methanolica. Any suitable vector may be used, e.g., YIp vectors, replicating episomal YEp vectors containing centromere sequences CEN and autonomously replicating sequences ARS.

Insect cells may be advantageous, e.g., for high efficiency protein expression. Exemplary insect host cells are those from Spodoptera frugiperda (e.g., Sf9 and Sf21 cell lines, and EXPRESS™ cells (Protein Sciences Corp., Meriden, Conn., USA)), Drosophila S2 cells, and Trichoplusia ni HIGH FIVE® Cells (Invitrogen, Carlsbad, Calif., USA). Where the host cells are Spodoptera frugiperda cells, the vector replicative strategy is typically based upon the baculovirus life cycle.

Exemplary mammalian host cells are COS1 and COS7 cells, NSO cells, Chinese hamster ovary (CHO) cells, NIH 3T3 cells, 293 cells, HEPG2 cells, HeLa cells, L cells, MDCK, HEK293, WI38, murine ES cell lines (e.g., from strains 129/SV, C57/13L6, DBA-1, 129/SVJ), K562, Jurkat cells, BW5147 and any other commercially available human cancer cell lines. Cells with K-ras^{G13D}, such as human colon cancer cell lines DLD-1 and HCT-116, and revertants thereof having a null mutation in the activated K-ras gene, can also be used. Other useful mammalian cell lines are well known and are available from the American Type Culture Collection (ATCC) (Manassas, Va., USA) and the National Institute of General medical Sciences (NIGMS) Human Genetic Cell Repository at the Coriell Cell Repositories (Camden, N.J., USA). As used herein, “mammalian host cells” includes those in the body of a subject, e.g., a human patient or other mammal.

Vectors intended for autonomous extrachromosomal replication in mammalian cells typically include a viral origin, such as the SV40 origin (for replication in cell lines expressing the large T-antigen, such as COS1 and COS7 cells), the papillomavirus origin, or the EBV origin for long term episomal replication (for use in, e.g., 293-EBNA cells, which constitutively express the EBV EBNA-1 gene product and adenovirus E1A). Vectors intended for integration, and thus replication as part of the mammalian chromosome optionally include an origin of replication functional in mammalian cells, e.g., the SV40 origin. Useful vectors also include vectors based on viruses such as lentiviruses, adenovirus, adeno-associated virus, vaccinia virus, parvoviruses, herpesviruses, poxviruses, Semliki Forest viruses, and retroviruses.

Plant cells also can be used for expression, with the vector replicon typically derived from a plant virus (e.g., cauliflower mosaic virus (CaMV); tobacco mosaic virus (TMV)) and selectable markers chosen for suitability in plants.

The invention relates to artificial chromosomes, e.g., bacterial artificial chromosomes (BACs), yeast artificial chromosomes (YACs), mammalian artificial chromosomes (MACs), and human artificial chromosomes (HACs), that contain the GP131 nucleic acid of interest.

Vectors often include elements that permit in vitro transcription of RNA from the inserted heterologous nucleic acid. Such vectors typically include a phage promoter, such as that from T7, T3, or SP6, flanking the nucleic acid insert. Often two different such promoters flank the inserted nucleic acid, permitting separate in vitro production of both sense and antisense strands.

B. Transcription Regulators for Expression Vectors

Expression vectors often include a variety of other genetic elements operatively linked to the protein-encoding heterologous nucleic acid insert. Examples of other such genetic elements are promoters and enhancer elements. Other elements are those that facilitate RNA processing, e.g., transcription termination signals, splicing signals and polyadenylation signals. Other elements are those that facilitate translation, e.g., ribosomal consensus sequences. Examples of other transcription control sequences are operators and silencers. Use of such expression control elements, including those that confer constitutive or inducible expression, and developmental or tissue-regulated expression are well-known in the art.

Constitutively active promoters include, without limitation, a CMV promoter, EF1α; retroviral LTRs, and SV40 early region.

Inducible promoters useful in this invention include, without limitation, a tetracycline-inducible promoter, a metallothionine promoter, the IPTG/lacI promoter system, the ecdysone promoter system, and the “lox stop lox” system for irreversibly deleting inhibitory sequences for translation or transcription. In some embodiments, a GP131 gene is placed between lox sites. Upon expression of the cre enzyme, the GP131 gene is deleted from the genome so that the GP131 activity is permanently eliminated.

Instead of inducible promoters, the activity of a GP131 protein also can be inducibly switched on or off by fusing the GP131 protein to, e.g., an estrogen receptor polypeptide sequence, where administration of estrogen or an estrogen analog (e.g., hydroxytamoxifen) will allow the correct folding of the GP131 polypeptide into a functional protein.

Tissue-specific promoters that can be used in driving expression of GP131 in animal models include, without limitation: a tyrosinase promoter or a TRP2 promoter in the case of melanoma cells and melanocytes; an MMTV or WAP promoter in the case of breast cells and/or cancers; a Villin or FABP promoter in the case of intestinal cells and/or cancers; a RIP promoter in the case of pancreatic beta cells; a Keratin promoter in the case of keratinocytes; a Probasin promoter in the case of prostatic epithelium; a Nestin or GFAP promoter in the case of CNS cells and/or cancers; a Tyrosine Hydroxylase, S100 promoter or neurofilament promoter in the case of neurons; the pancreas-specific promoter described in Edlund et al. Science 230:912-916 (1985); a Clara cell secretory protein promoter in the case of lung cancer; and an Alpha myosin promoter in the case of cardiac cells.

Developmentally regulated promoters also can be used. They include, without limitation, the murine hox promoters (Kessel and Gruss, Science 249:374-379 (1990)) and the α-fetoprotein promoter (Campes and Tilghman, Genes Dev. 3:537-546 (1989)).

C. Expression Vectors Encoding Peptide Tags

Expression vectors can be designed to fuse the expressed polypeptide to small protein tags that facilitate purification and/or visualization. Many such tags are known and available. Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as luciferase or those that have a green fluorescent protein (GFP)-like chromophore, and fusions for use in two hybrid selection systems.

For secretion of expressed proteins, a wide variety of vectors are available which include appropriate sequences that encode secretion signals, such as leader peptides. Vectors designed for phage display, yeast display, and mammalian display, for example, target recombinant proteins using an N-terminal cell surface targeting signal and a C-terminal transmembrane anchoring domain.

A wide variety of vectors now exist that fuse proteins encoded by heterologous nucleic acids to the chromophore of the substrate-independent, intrinsically fluorescent green fluorescent protein from Aequorea victoria (“GFP”) and its many color-shifted and/or stabilized variants.

For long-term, high-yield recombinant production of the proteins, protein fusions, and protein fragments of the present invention, stable expression is preferred. Stable expression is readily achieved by integration into the host cell genome of vectors (preferably having selectable markers), followed by selection for integrants.

III. GP131 Proteins, Polypeptides and Fragments

The present invention relates to GP131 proteins and various fragments suitable for use, e.g., as antigens, biomarkers for diseases, and in therapeutic compositions. The invention also relates to fusions of GP131 polypeptides to heterologous polypeptides or other moieties.

A. GP131 Polypeptides of Particular Sequences

The invention relates to an isolated GP131 polypeptide (SEQ ID NO: 1), optionally containing one or more conservative amino acid substitutions. The invention also relates to fragments of the GP131 polypeptide, particularly fragments having at least 5, 6, 8, or 15 amino acids of SEQ ID NO: 1. Larger fragments of at least 20, 25, 30, 35, 50, 75, 100, 150 or more amino acids are also useful, and at times preferred. The GP131 fragments of the invention may be continuous portions of the native GP131 protein. However, it will be appreciated that knowledge of the GP131 gene and protein sequences permits recombining of various domains that are not contiguous in the native GP131 protein.

B. Fusion Proteins and Other Protein Conjugates

This invention also relates to fusions of GP131 polypeptides to heterologous polypeptides. As used herein, “fusion” means that the GP131 polypeptide is linearly contiguous to the heterologous polypeptide in a peptide-bonded polymer of amino acids or amino acid analogues. As used herein, “heterologous polypeptide” means a polypeptide that does not naturally occur in contiguity with the GP131 fusion partner. The fusion polypeptide can consist entirely of a plurality of fragments of the GP131 protein in altered arrangement. In such a case, any of the GP131 fragments can be considered heterologous to the other GP131 fragments in the fusion protein.

The heterologous polypeptide included within the fusion protein is at least 6 amino acids in length, often at least 8 amino acids in length, and preferably, at least 15, 20, and 25 amino acids in length. The heterologous sequences can target the GP131 polypeptide to a selected cell by binding to a cell surface receptor, prolong the serum life of the GP131 polypeptide (e.g., an IgG Fc region), make the GP131 polypeptide detectable (e.g., a luciferase or a green fluorescent protein), facilitate purification (e.g., His tag, FLAG, etc.), facilitate secretion of recombinantly expressed proteins (e.g., into the periplasmic space or extracellular milieu for prokaryotic hosts, into the culture medium for eukaryotic cells, through incorporation of secretion signals and/or leader sequences). Other useful protein fusions include fusions that permit use of the protein of the present invention as bait in a yeast two-hybrid system, fusions that display the encoded protein on the surface of a phage or cell, and fusions to intrinsically detectable proteins, such as fluorescent or light-emitting proteins.

The proteins and protein fragments also can be fused to protein toxins, such as Pseudomonas exotoxin A, diphtheria toxin, shiga toxin A, anthrax toxin lethal factor, ricin, or other toxic moieties in order to effect specific ablation of cells that bind or take up the proteins.

C. Other Modifications of the Polypeptides

The polypeptides can be composed of natural amino acids linked by native peptide bonds, or can contain any or all of nonnatural amino acid analogues, nonnative bonds, and post-synthetic (post-translational) modifications, either throughout the length of the polypeptide or localized to one or more portions thereof. However, the range of such nonnatural analogues, nonnative inter-residue bonds, or post-synthesis modifications will be limited to those that do not interfere with the biological function of the polypeptide.

Techniques for incorporating non-natural amino acids during solid phase chemical synthesis or by recombinant methods are well established in the art. For instance, D-enantiomers of natural amino acids can readily be incorporated during chemical peptide synthesis: peptides assembled from D-amino acids are more resistant to proteolytic attack; incorporation of D-enantiomers can also be used to confer specific three dimensional conformations on the peptide. Other amino acid analogues commonly added during chemical synthesis include ornithine, norleucine, phosphorylated amino acids (typically phosphoserine, phosphothreonine, phosphotyrosine), L-malonyltyrosine, a non-hydrolyzable analog of phosphotyrosine (Kole et al., Biochem. Biophys. Res. Com. 209:817-821 (1995)), and various halogenated phenylalanine derivatives.

The isolated GP131 polypeptides can also include non-native inter-residue bonds, including bonds that lead to circular and branched forms. The isolated GP131 polypeptides can also include post-translational and post-synthetic modifications, either throughout the length of the protein or localized to one or more portions thereof. For example, when produced by recombinant expression in eukaryotic cells, the isolated polypeptide can include N-linked and/or O-linked glycosylation, the pattern of which will reflect both the availability of glycosylation sites on the protein sequence and the identity of the host cell. Further modification of glycosylation pattern can be performed enzymatically. As another example, recombinant polypeptides of the invention may also include an initial modified methionine residue, in some cases resulting from host-mediated processes.

Amino acid analogues having detectable labels are also usefully incorporated during synthesis to provide a labeled polypeptide (e.g., biotin, various chromophores, or fluorophores). The GP131 polypeptides of this invention can also usefully be conjugated to polyethylene glycol (PEG). PEGylation increases the serum half life of proteins administered intravenously for replacement therapy.

D. Purification of the Polypeptides

Production of the isolated polypeptides optionally can be followed by purification from the producing cells. Producing cells include, without limitation, recombinant cells overexpressing the polypeptides, naturally occurring cells (e.g., cancer cells) overxpression the polypeptides, or established cancer cell lines overexpressing the polypeptides. If purification tags have been fused through use of an expression vector that appends such tags, purification can be effected, at least in part, by means appropriate to the tags, such as use of immobilized metal affinity chromatography for polyhistidine tags. Other techniques common in the art include ammonium sulfate fractionation, immuno-precipitation, fast protein liquid chromatography (FPLC), high performance liquid chromatography (HPLC), and preparative gel electrophoresis. Purification of chemically-synthesized peptides can readily be effected, e.g., by HPLC.

Accordingly, the isolated GP131 proteins may be in pure or substantially pure form. A purified protein is an isolated protein that is present at a concentration of at least 95%, as measured on a mass basis (w/w) with respect to total protein in a composition. Such purities can often be obtained during chemical synthesis without further purification, as, e.g., by HPLC. Purified proteins can be present at a concentration (measured on a mass basis with respect to total protein in a composition) of 96%, 97%, 98%, and even 99%. The proteins of the present invention can even be present at levels of 99.5%, 99.6%, and even 99.7%, 99.8%, or even 99.9% following purification.

Although high levels of purity are preferred when the isolated proteins are used as therapeutic agents, the isolated proteins are also useful at lower purity. For example, partially purified proteins can be used as immunogens to raise antibodies in laboratory animals.

The isolated proteins are generally used in substantially purified form. As used herein, “substantially purified protein” means a protein present at a concentration of at least 70%, measured on a mass basis with respect to total protein in a composition. Usefully, the substantially purified protein is present at a concentration, measured on a mass basis with respect to total protein in a composition, of at least 75%, 80%, or even at least 85%, 90%, 91%, 92%, 93%, 94%, 94.5% or even at least 94.9%.

In preferred embodiments, the purified and substantially purified proteins are in compositions that lack detectable ampholytes, acrylamide monomers, bis-acrylamide monomers, and polyacrylamide.

E. Exemplary Uses of GP131 Polypeptides

1. Therapeutic Use

Certain fragments of at least 8, 9, 10 or 12 contiguous amino acids are also useful as competitive inhibitors of binding of the entire protein, or a portion thereof, to its ligand. Thus, such fragments can be used as anti-cancer agents to reduce the activity of GP131.

2. Epitope Mapping

Fragments of at least six contiguous amino acids are useful in mapping B cell and T cell epitopes of the reference protein. See, e.g., Geysen et al., “Use of peptide synthesis to probe viral antigens for epitopes to a resolution of a single amino acid,” Proc. Natl. Acad. Sci. USA 81:3998-4002 (1984) and U.S. Pat. Nos. 4,708,871 and 5,595,915. Because the fragment need not itself be immunogenic, part of an immunodominant epitope, nor even recognized by native antibody, to be useful in such epitope mapping, all fragments of at least six amino acids of the proteins of the present invention have utility in such a study.

3. Immunogens

Fragments of at least eight contiguous amino acids, often at least fifteen contiguous amino acids, have utility as immunogens for raising antibodies that recognize GP131 proteins or as vaccines for GP131-mediated diseases such as cancers.

The GP131 proteins, fragments, and fusions of the present invention can usefully be attached to a substrate. When bound to a substrate, the polypeptides can be used to detect and quantify antibodies, e.g., in serum, that bind specifically to the immobilized protein.

IV. Antibodies and Antibody-Producing Cells

A. General Consideration

The invention relates to antibodies that bind specifically to the GP131 polypeptides. The antibodies can be specific for linear epitopes, discontinuous epitopes, or conformational epitopes of such polypeptides, either as present on the polypeptide in its native conformation or as present on the polypeptides when denatured, e.g., by solubilization in SDS. In some embodiments, the antibodies, both polyclonal and monoclonal, bind specifically to a polypeptide having an amino acid sequence presented in SEQ ID NO: 1.

As used herein, an “antibody” means a full antibody, e.g., an antibody comprising two heavy chains and two light chains, or to an antigen-binding fragment of a full antibody. Such fragments include, but are not limited to, those produced by digestion with various proteases, those produced by chemical cleavage and/or chemical dissociation, and those produced recombinantly, so long as the fragment remains capable of specific binding to an antigen. Among these fragments are Fab, Fab′, F(ab′)₂ and single chain Fv (scFv) fragments.

An antibody can be a murine or hamster antibody or a homolog thereof, or a fully human antibody. An antibody also can be a humanized antibody, a chimeric antibody, an antibody fusion, an diabody, an intrabody, or a single-chained antibody. An antibody can be of any isotype and subtype, for example, IgA (e.g., IgA1 and IgA2), IgG (e.g., IgG1, IgG2, IgG3 and IgG4), IgE, IgD, IgM, wherein the light chains of the immunoglobulin may be of type kappa or lambda. While the useful antibodies are generally monoclonal, polyclonal antibodies from mice, rabbits, turkeys, or sheep may also be used.

Typically, the affinity or avidity of an antibody (or antibody multimer, as in the case of an IgM pentamer) will be at least about 1×10⁻⁶ molar (M), typically at least about 5×10⁻⁷ M, usefully at least about 1×10⁻⁷ M, with affinities and avidities of at least 1×10⁻⁸ M, 5×10⁻⁹ M, and 1×10 ⁻¹⁰ M proving especially useful.

B. Moieties Conjugated to the Antibodies

The antibodies are useful in a variety of in vitro immunoassays, such as Western blotting and ELISA, in isolating and purifying GP131 proteins (e.g., by immunoprecipitation, immunoaffinity chromatography, or magnetic bead-mediated purification). The antibodies are also useful as modulators (i.e., antagonists or agonists) of a GP131 protein in vivo to modulate the protein's interaction with its natural ligand. The antibodies can also be used to conjugate to cytotoxic reagents for site-specific delivery.

The new antibodies can be variously associated with moieties appropriate for their uses. When the antibodies are used for immunohistochemical staining of tissue samples, the moieties can be an enzyme that catalyzes production and local deposition of a detectable product. Exemplary enzymes are alkaline phosphatase, β-galactosidase, glucose oxidase, horseradish peroxidase (HRP), and urease. The antibodies also can be labeled using colloidal gold. When the antibodies are used for flow cytometric detection and scanning laser cytometric detection, they can be labeled with fluorophores. For secondary detection using labeled avidin, streptavidin, captavidin or neutravidin, the antibodies of the present invention can usefully be labeled with biotin. When the antibodies of the present invention are used, e.g., for western blotting, they can usefully be labeled with radioisotopes. When the antibodies are to be used for in vivo diagnoses, they can be rendered detectable by conjugation to MRI contrast agents, such as radioisotopic labeling or gadolinium diethylenetriaminepentaacetic acid (DTPA).

The antibodies also can be conjugated to toxic agents so as to direct the agents to a tumor site. By way of example, the antibody is conjugated to Pseudomonas exotoxin A, diphtheria toxin, shiga toxin A, anthrax toxin lethal factor, or ricin. See, e.g., Hall (ed.), Immunotoxin Methods and Protocols (Methods in Molecular Biology, Vol 166), Humana Press (2000) (ISBN:0896037754); and Frankel et al. (eds.), Clinical Applications of Immunotoxins, Springer-Verlag New York, Incorporated (1998) (ISBN:3540640975). Small molecule toxins such as calicheamycin or chemotherapeutic agents can also be delivered via chemical conjugation to the antibodies. The antibodies may also be used to deliver DNA to the tumor site as gene therapy to inhibit or otherwise modify the behavior of the tumor, e.g., to deliver an antisense reagent to the GP131 gene.

For some uses, the antibodies can be bound to a substrate via a linker moiety. For example, the antibodies can be conjugated to filtration media, such as NHS-activated Sepharose or CNBr-activated Sepharose for immunoaffinity chromatography. The antibodies also can be attached to paramagnetic microspheres, by, e.g., biotin-streptavidin interaction. The microsphere then can be used for isolation of cells that express or display the above-described proteins. The antibodies also can be attached to the surface of a microtiter plate for ELISA.

V. Pharmaceutical Compositions

As a protein involved in tumor genesis, development and/or maintenance, GP131 is a suitable therapeutic target for treating neoplasia, hyperplasia, malignant cancers, or any other hyperproliferative conditions. For example, the GP131 gene can be a target in cancers of the skin, lung, prostate, breast, colorectal tissue, liver, pancreas, brain, testis, ovary, uterus, cervix, kidney, thyroid, bladder, esophagus, and blood.

Accordingly, the invention relates to pharmaceutical compositions comprising GP131 nucleic acids, proteins, and antibodies, as well as mimetics, agonists, antagonists, or modulators of GP131 activity, and methods of using them to prevent or treat (i.e., ameliorate, mitigate, alleviate, slow, or inhibit) tumor growth, angiogenesis, metastasis or any other inappropriate cell proliferation.

Inhibitors of GP131 also can be administered in combination with one or more other therapeutic agents, for improved cancer treatment. Other therapeutic agents suitable for co-administration with a GP131 inhibitor include, for example, an anti-angiogenic agent, an anti-metastatic agent, or an agent that creates a hypoxic environment. Chemotherapeutic agents that can be co-administered with inhibitors of GP131 include folate antagonists, pyrimidine and purine antimetabolites, alkylating agents, platinum antitumor compounds, DNA interchelators, other agents that induce DNA damage, microtubule targeting products, small molecule inhibitors of protein kinases and biological inhibitors of growth factor receptors. The GP131 inhibitor and additional therapeutic agent(s) may be used concurrently or sequentially. In some embodiments, the subject is pre-treated with one or more agents, followed by treatment with a GP131 inhibitor.

The ability of tumor cells to detach from the primary site and produce metastases in a distant organ is due to the survival and growth of a unique subpopulation of cells with metastatic properties. Tumor growth and metastasis are angiogenesis-dependent. inhibition of angiogenesis will generate a hypoxic environment in the tumor, forcing tumor cells to become more dependent upon the glycolytic pathway for energy generation. Accordingly, preventing angiogenesis in combination with inhibiting GP131 is a promising therapeutic strategy. Angiogenesis inhibitors, e.g. angiostatin, endostatin, avastin or Regeneron's VEGF trap technology, can be used in combination with GP131 inhibitors as an effective anti-cancer therapy. Such a combination can be expected to have a synergistic effect. This may also allow the use of a lower dose of GP131 inhibitor or anti-angiogenic agent or both in chemotherapy. This is desirable because it is likely to cause less toxicity in patients. In addition, the use of combinations of therapeutic agents may circumvent drug resistance problems.

GP131 inhibitors can also be used in combination with agents that create a hypoxic environment to enhance the effect of GP131 inhibitor. Hypoxia, i.e., lack of oxygen, plays a fundamental role in many pathologic processes. In response to hypoxia, mammalian cells activate and express multiple genes. Tumor cells may respond to hypoxia by diminishing their proliferative rates, thereby leaving the cells viable but nonproliferating. Some transformed cell lines can also undergo apoptosis in extreme hypoxia and an acidic environment. Similar to inhibitors of angiogenesis, other agents that induce a hypoxic environment may sensitize tumor cells to inhibition of GP131 and use of hypoxia inducing agents in combination with inhibiting GP131 is therefore another promising therapeutic strategy.

An increase in apoptosis (programmed cell death) has been associated with a decrease in tumor proliferation. Tumor necrosis factor-related apoptosis-inducing ligand (TRAIL) induces apoptosis in several human tumors. A number of cell regulatory pathways such as Rb/E2F pathway, the c-Myc transcription factor, and the Ras signaling molecule have also been shown to control not only cell proliferation but also pathways leading to apoptosis. Further, a combination of GP131 inhibitor with reagents that activate additional apoptotic signals, or inhibit survival signals, can also be used for cancer therapy. Survival signals that has recently been shown to modulate apoptotic signaling include the focal adhesion kinase (FAK), the phosphinositol 3′ kinase (PI3′K), and protein kinase B (PKB, also known as Akt).

A composition of the invention typically contains from about 0.1 to 90% by weight (such as 1 to 20% or 1 to 10%) of a therapeutic agent of the invention in a pharmaceutically accepted carrier. Solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch glycolate, and alginic acid. Tablet binders that can be used include acacia, methylcellulose, sodium carboxymethylcellulose, polyvinylpyrrolidone (Povidone™), hydroxypropyl methylcellulose, sucrose, starch, and ethylcellulose. Lubricants that can be used include magnesium stearates, stearic acid, silicone fluid, talc, waxes, oils, and colloidal silica.

Liquid formulations of the compositions for oral administration prepared in water or other aqueous vehicles can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). Physiologically acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically acceptable oil base, such as an ester of a long chain fatty acid (e.g., ethyl oleate).

A topical semi-solid ointment formulation typically contains a concentration of the active ingredient from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. The optimal percentage of the therapeutic agent in each pharmaceutical formulation varies according to the formulation itself and the therapeutic effect desired in the specific pathologies and correlated therapeutic regimens.

Pharmaceutical formulation is a well-established art, and is further described in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X), the disclosures of which are incorporated herein by reference in their entireties. Conventional methods, known to those of ordinary skill in the art of medicine, can be used to administer the pharmaceutical formulation(s) to the patient.

Typically, the pharmaceutical formulation will be administered to the patient by applying to the skin of the patient a transdermal patch containing the pharmaceutical formulation, and leaving the patch in contact with the patient's skin (generally for 1 to 5 hours per patch). Other transdermal routes of administration (e.g., through use of a topically applied cream, ointment, or the like) can be used by applying conventional techniques. The pharmaceutical formulation(s) can also be administered via other conventional routes (e.g., parenteral, subcutaneous, intrapulmonary, transmucosal, intraperitoneal, intrauterine, sublingual, intrathecal, or intramuscular routes) by using standard methods. In addition, the pharmaceutical formulations can be administered to the patient via injectable depot routes of administration such as by using 1-, 3-, or 6-month depot injectable or biodegradable materials and methods.

Regardless of the route of administration, the therapeutic protein or antibody agent typically is administered at a daily dosage of 0.01 mg to 30 mg/kg of body weight of the patient (e.g., 1 mg/kg to 5 mg/kg). The pharmaceutical formulation can be administered in multiple doses per day, if desired, to achieve the total desired daily dose. The effectiveness of the method of treatment can be assessed by monitoring the patient for known signs or symptoms of a disorder.

The pharmaceutical compositions of the invention may be included in a container, package or dispenser alone or as part of a kit with labels and instructions for administration. These compositions can also be used in combination with other cancer therapies involving, e.g., radiation, photosensitizing compounds, anti-neoplastic agents and immunotoxics.

VI. GP131-Related Animals

A. General Consideration

This invention provides genetically modified non-human mammals at least some of whose somatic and germ cells contain one of the above-described GP131-coding nucleic acid of this invention (including both heterozygotes and homozygotes). Such mammals can be used to study the effect of the GP131 gene on tumorigenicity and tumor development, to study the role of GP131 on normal tissue development and differentiation, to identify via array CGH regions of the genome whose amplification or deletion is correlated with GP131 status, and to screen for and establish toxicity profiles of anti-cancer drugs. This invention also provides genetically modified non-human mammals with targeted disruption of one or both copies of the endogenous GP131 gene. Also included are chimeric mammals.

B. Inducible Cancer Model

This invention provides an inducible cancer model to study tumor biology and to screen for anti-cancer drugs. In some embodiments, the inducible cancer model is a mouse whose genome has been modified to include: (a) an expression construct comprising a GP131 gene linked operably to an inducible promoter, and (b) a genetic mutation that causes the mouse to have greater susceptibility to cancer than a mouse not comprising said genetic mutation. Expression of the GP131 gene leads to formation of cancer in the mouse. The cancer regresses when expression of the GP131 gene is reduced. Mutations that render the mammal more susceptible to cancer include disabling mutations in a tumor suppressor gene (e.g., INK4a), disabling mutations in a DNA repair gene (e.g., MSH2), and activating mutations in an oncogene (e.g., myc and ras). Such testing also can be carried out in cells (e.g., human cells) that are engineered to contain an inducible oncogene and endowed with tumorigenic capacity by the presence of an appropriate combination of oncogenes, tumor suppressor genes, and/or telomerase.

In one particular embodiment, the mammal's genome comprises (i) a first expression construct containing a gene encoding a reverse tetracycline transactivator operably linked to a promoter, such as any tissue or cell type-specific promoter or any general promoter, and (ii) a second expression construct containing the GP131 gene operably linked to a promoter that is regulated by the reverse tetracycline transactivator and tetracycline (or a tetracycline analogue, for example, doxycycline). The mammal is observed with and without administration of tetracycline (or analogue thereof) for the development, maintenance, or progression of a tumor that is tetracycline-dependent. Other inducible systems such as those described above also can be employed.

This animal model can be used to determine the efficacy of a candidate compound in preventing or treating cancer. This method involves administering to the mammal a candidate compound and observing the effect of the compound on tumor development, maintenance, angiogenesis and/or progression in the mammal. Regression and/or reduction of tumor size in the presence of the compound indicates effectiveness of the compound. Similarly, the effect of a candidate compound on the level of GP131 mRNA, protein, or activity in the mammal or cell lines derived from the mammal (or cell lines transfected with the gene) can be used to identify the candidate as an agonist or antagonist. The ability to compare the effect of a test compound to that of genetically switching off the inducible oncogene in this system allows the identification of surrogate markers that are predictive of the clinical response to the compound. The inducible model can be used to determine whether a compound can eradicate minimal residual tumor. Normally in the inducible model, a tumor regresses when the GP131 gene is switched from “on” to “off” via the inducible promoter. But if a compound can eradicate minimal residual tumor, switching the gene back on after administration of the compound will not bring back the tumor.

The animal model also can be used to identify other cancer-related elements. To do this, a detailed expression profile of gene expression in tumors undergoing regression or regrowth due to the inactivation or activation of the GP131 transgene is established. Techniques used to establish the profile include the use of suppression subtraction (in cell culture), differential display, proteomic analysis, serial analysis of gene expression (SAGE), and expression/transcription profiling using cDNA and/or oligonucleotide microarrays. Then, comparisons of expression profiles at different stages of cancer development can be performed to identify genes whose expression patterns are altered.

This animal model can also be used to identify molecular surrogates of GP131 in another manner. To do this, the expression of GP131 gene is eliminated by null mutation, and another round of MaSS screening is performed using retroviral integration, cDNA complementation, or the genetic supressor elements (GSE) method. Genes whose activation results in transformation of the cells are likely to be in a tumorigenic pathway related to GP131.

The animal model also can be used to identify surrogate biomarkers for diagnosis or for following disease progression in patients. The biomarkers can be identified based on the differences between the expression profiles of the “on” and “off” states in the animal model. Blood or urine samples from the animal can be tested with ELISAs or other assays to determine which biomarkers are released from the tumor into circulation during tumor genesis, maintenance, or regression (when GP131 is turned off). These biomarkers are particularly useful clinically in following disease progression post anti-GP131 therapy.

VII. Cancer Diagnostics

Since GP131 activity is up-regulated in tumor cells, one can use GP131 as a marker in diagnosing cancer or any other abnormal hyperplasia conditions. To do this, the above-described nucleic acid probes or antibodies are used to quantify the expression level of GP131 in a tissue sample. An increase in that level relative to control indicates cancerous, neoplastic, or hyperplastic pathology of the tissue sample. This type of test can be performed using conventional techniques such as RT-PCR, ribonuclease protection assays, in situ hybridization, Northern blot analysis, FISH, CGH, array CGH, SKY, and immunohistochemistry.

Because up-regulation of GP131 is generally associated with a malignant state, a GP131 protein or GP131 protein fragments may be found to be elevated in a tissue sample (e.g., blood or urine) of cancer patients relative to that of normal individuals. This elevation can be detected by, e.g., specific ELISAs, radioimmunoassays, or protein chip assays. Such tests may not only be useful for diagnosis of GP131-related diseases such as cancers, but also for monitoring the progress of therapy using GP131 inhibitors.

### EXAMPLES

The invention is further illustrated by the following examples These examples are provided for illustrative purposes only, and are not to be construed as limiting the scope or content of the invention in any way.

### Example 1

### MaSS Screening Identification of the Gene

This example describes the procedures for identifying the GP131 gene by MaSS screening.

a. Retroviral Infection of Tumor Cells

Mo-MuLV producer cell line TMJ (NIH3T3 based cell line) was plated to the required number of plates (100 mm). These cells were cultured and maintained in RPMI media with 10% FBS. For viral production, TMJ cells were fed with 4-5 ml of fresh culture media, and culture supernatant was harvested 8-12 hours later. The supernatant was filtered through a 0.45 μM filter.

Meanwhile, doxycycline-dependent, RAS-induced melanoma cells such as R545 cells were maintained in RPMI media with 10% fetal calf serum in the presence of doxycycline (2 μg/ml). At approximately 18-24 hrs after plating or when the plates were 70-80% confluent, the R545 cells were infected with the filtered viral supernatant in the presence of polybrene (6-8 μg/ml). From this point on, the R545 cells were maintained in the absence of doxycycline.

Eighteen hours after infection, infected R545 cells were trypsinized, rinsed and resuspended in Hanks' Balanced Salt Solution. Cell suspensions were kept on ice and the handling time after trypsinization was kept to a minimum. About 1×10⁶ cells were injected onto the flank of SCID mice fed with water without doxycycline. The animals were observed for tumor development. Control animals were similarly injected with 1×10⁶ uninfected R545 cells. Tumors typically developed after approximately 21 days. Tumors were harvested and tumor tissues were immediately snap-frozen in LN₂.

b. Inverted Polymerase Chain Reaction

DNA was isolated from tumor tissues using the PUREGENE DNA isolation kit. 10 μg of genomic DNA was digested to completion with either BamHI or SacII and the reaction was terminated by incubation at 65° C. for 20 minutes. The digested samples were self-ligated in a diluted 600 μl reaction volume using 4000 U of high concentration T4 Ligase (NEB, Cat. # M0202M). The ligation was performed overnight to 24 hrs at 16° C. The ligated DNA was precipitated with ethanol and dissolved in 40 μl of sterile water. The ligated DNA was then serially diluted to 1:10 and 1:100 ratios and subjected to inverted polymerase chain reaction (IPCR).

The PCR reaction mix had a total volume of 50 μl and contained 1 μl of the ligated DNA, 25 nmol of each dNTP, 10 pmol each of the forward and reverse primers, 1× Buffer 2, and 2.5 U of Enzyme Mix in the EXPAND Long Template PCR System (Roche). Amplification was performed as follows: 92° C. for 2 min, then 10 cycles of (92° C. for 10 sec, 63° for 30 sec, 68° C. for 15 min), then 20 cycles of (92° C. for 10 sec, 63° C. for 30 sec, 68° C. for 15 min, and a 20 sec auto extension), and a final extension step at 68° C. for 30 min (TETRAD Thermocycler, MJ Research). The primer sets used in IPCR (all of them targeting the retroviral LTRs) were:

(SEQ ID NO: 10)

S5′1F: GAGGCCACCTCCACTTCTGAGAT;

(SEQ ID NO: 11)

S5′1R: CTCTGTCGCCATCTCCGTCAGA;

(SEQ ID NO: 12)

S5′2F: CAUCAUCAUCAUCCTGCCCCCTCTCCCATAGTGT;

(SEQ ID NO: 13)

S5′2R: CUACUACUACUAGGCGTTACTGCAGTTAGCTGGCT;

(SEQ ID NO: 14)

S3′1F: GGCTGCCATGCACGATGACCTT;

(SEQ ID NO: 15)

S3′1R: CGGCCAGTACTGCAACTGACCAT;

(SEQ ID NO: 16)

S3′2F: CUACUACUACUAGGGAGGGTCTCCTCAGAGTGATT;

(SEQ ID NO: 17)

S3′2R: CAUCAUCAUCAUGGAAAGCCCGAGAGGTGGT;

(SEQ ID NO: 18)

B3′1R: CGGGAAGGTGGTCGTCGGTCT;
and

(SEQ ID NO: 19)

B3′2R: CAUCAUCAUCAUGGGGCCCCGAGTCTGTAATTT.

For BamHI 5′ cloning, primary PCR was done by using S5′1F and S5′1R followed by nested PCR using S5′2F and S5′2R. For BamHI 3′ cloning, primary PCR was done by using S3′1F and B3′1R followed by nested PCR using S3′2F and B3′2R. For SacII 5′ cloning, primary PCR was done by using S5′1F and S5′1R followed by nested PCR using S5′2F and S5′2R. For SacII 3′ cloning, primary PCR was done by using S3′1F and S3′1R followed by nested PCR using S3′2F and S3′2R.

The PCR products were resolved on 1% agarose gel. Individual bands were excised and purified using the QIAGEN Gel Extraction kit. The purified DNA was dissolved in 30 μl of sterile water and subjected to direct DNA sequencing.

c. Identification of Candidate Genes

The site of retroviral integration into the mouse genome was mapped for all IPCR sequences as follows. Retroviral leader sequences were trimmed from the raw sequences of IPCR products, and homology searches for the trimmed sequences were performed in the NCBI MGSCV3 database by using the BLAST software program. BLAST hits were analyzed and recurrent sites of integration in multiple mouse tumors were identified. Recurrence was defined as 2 or more integrations within a 10 kb region. To identify genes whose expression was affected by the retroviral integration, NCBI MapView was used to identify the site of each recurrent retroviral integration onto the mouse genome. Genes immediately neighboring the site were identified by using the MGSCV3 Gene map. These genes were defined as candidate cancer-related genes because in the vast majority of cases, MuLV integration affects the most proximal genes. When the integration occurred within a gene, that gene was deemed the best candidate as the target for the effects of retroviral integration.

### Example 2

### Expression in Human Tumors and Tumor Cell Lines

This example describes the protocols for expressing the candidate gene identified above in human cancer cells.

Primer pairs for each human gene were designed as described for the mouse gene. Expression of each candidate gene was assessed in a panel of 31 human cancer cell lines and 47 human primary tumors by using real-time reverse transcription PCR. The forward and reverse primers were 5′-cacatggaggatgaggacag-3′ (SEQ ID NO: 20) and 5′-gagtctggcttcggattctc-3′ (SEQ ID NO: 21), respectively. RNA was prepared from the cells using QIAGEN RNEASY mini-prep kits and QIAGEN RNEASY maxi-prep columns. RNA preparations were treated with DNase during column purification according to manufacturer's instructions. Expression of each gene was determined in triplicate for all tumors and cell lines using SYBR green-based real-time PCR. To do this, 2×SYBR green PCR master mix (ABI) was mixed with the MULTISCRIBE reverse transcriptase (ABI) and RNase inhibitor (ABI). Forward and reverse primers were added at ratios previously optimized for each gene using control human reference RNA (Stratagene). 50 ng of RNA template was used per reaction, and the reactions were performed in a total volume of 20 μl. Real-time quantification was performed using the ABI 7900HT and SDS2.0 software. RNA loading was normalized for β-actin and 18S rRNA. RNA quantity was determined relative to human universal reference RNA (Stratagene) to permit run-to-run comparisons.

### Example 3

### RNAi Inhibition of Human Cancer Cell Lines

This example describes the protocols used to inhibit the expression of the candidate gene in human cancer cells by RNA interference (RNAi).

a. Generation of siRNA

Double-stranded siRNA oligonucleotides were designed using the OligoEngine siRNA design tool (http://www.oligoengine.com). HPLC purified siRNA oligonucleotides were incubated at 95° C. for 1 min, 37° C. for 1 hr, and room temperature for 30 min. siRNAs were stored at −20° C. prior to use.

Human cancer cell lines were transfected with siRNA using the Oligofectamine reagent from Invitrogen. Transfected cells were allowed to grow for at least 24 hours. The cells were then trypsinized and reseeded for growth curve analysis or growth in soft agar.

b. Growth Curves

To create growth curves, early passage cell lines were seeded at 2×10⁴ cells per well in 12-well plates. At 6 (Day 0), 24 (Day 1), 48 (Day 2), 72 (Day 3), 96 (Day 4) and 120 hours (Day 5) after plating, duplicate plates of cells were washed, fixed in 10% buffered formalin, and stained with crystal violet for 30 min at room temperature. Stained cells were washed in double distilled water, and stain was extracted using 10% acetic acid. Absorbance of the extracted stain was read at 590 nm. The mean absorbance per well of duplicate cultures was determined.

c. Soft Agar Assay

One day prior to seeding cells for assay, 6-well plates containing bottom agar were prepared. The bottom agar was made of 0.7% agarose (SeaKem GTG agarose) and 1×DME plus 10% fetal bovine serum. The next day, cells were seeded into each well by adding 5 ml of a mixture containing 1×10⁴ cells in 0.32% agarose and 1×DME plus 10% FBS. The cell mixture was allowed to solidify at room temperature for 30 min. The plates were then incubated at 37° C. in a 5% CO₂ atmosphere for 1 to 14 days. Colony formation was analyzed and photographs taken at various time points.

d. Sequence of siRNAs for Target Genes

The sense sequence of three independent siRNAs that inhibit expression of GP131 are shown below.

(SEQ ID NO: 22)

GP-131-1 5′ GATCATCGCCGTTGAGGAAtt 3′

(SEQ ID NO: 23)

GP-131-2 5′ CAAGGCAAGCGGATATATGtt 3′

(SEQ ID NO: 24)

GP-131-3 5′ ACGGACTGCGTGTGTTACTtt 3′

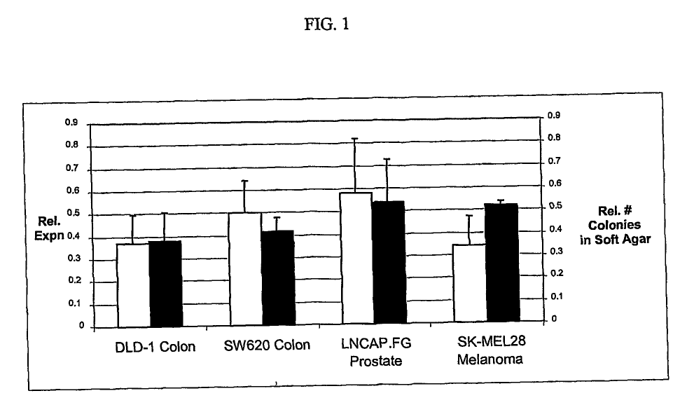

The effect of transfecting GP131-specific siRNA in human cancer cell lines DLD-1 (colon), SW620 (colon), LNCAP.FG (prostate) and SK-MEL28 (melanoma) was compared to the effect of transfecting a control siRNA that targets firefly luciferase. The data are summarized in FIG. 1. In the treated cells, GP115 expression was reduced to levels ranging from approximately 25% of the control value to approximately 51% of the control value. Cell growth, as represented by the number of colonies present in the agar at the end of 10 days, was reduced to levels ranging from approximately 8% of the control value to approximately 51% of the control value (FIG. 1).

e. Short hairpin RNA

The following DNA sequences were used to encode short hairpins RNAs that act through RNA interference mechanisms to knockdown expression of GP131. The sense and antisense sequences are joined by a loop of sequence gcttcctgtcac and are terminated with the following termination sequence containin an EcoRI restriction site: tttttgaattcc. These sequences are combined and expressed under the control of a regulatable U6 promoter to encode the short-hairpin RNA that knocks down or silences expression of the specific target gene. Stable cell lines were generated that express a tetracycline regulated transactivator and a U6 promoter under the control of the tetracycline response element driving expression of the shRNA. Addition of tetracycline or doxycycline to the cells in vivo or in vitro promotes expression of the shRNA and knock-down of expression of the GP131 gene.

TABLE 1
Exemplary shRNA sequences

| | | | | |
|---|---|---|---|---|
| shRNA 25 mer | | | | |
| position based on | | % | | |
| CDS or full mRNA | antisense 25 mer region | silencing | antisense 21 mer | sense 21 mer DNA |

| | | | | |
|---|---|---|---|---|
| GP131 | ucuuucauauauccgcuugccuugu | 32.1 | uuucauauauccgcuugcc | ggcaagcggatatatgaaa |
| 432-456 | (SEQ ID NO: 25) | | (SEQ ID NO: 31) | (SEQ ID NO: 37) |
| GP131 | gcuuccuaucccuuggagaucccac | 55.3 | uuccuaucccuuggagauc | gatctccaagggataggaa |
| 1012-1036 | (SEQ ID NO: 26) | | (SEQ ID NO: 32) | (SEQ ID NO: 38) |
| GP131 | ccauacagugcuuucuucugcuccu | 78.7 | auacagugcuuucuucugc | gcagaagaaagcactgtat |
| 1088-1112 | (SEQ ID NO: 27) | | (SEQ ID NO: 33) | (SEQ ID NO: 39) |
| GP131 | cauugauggccagaaacuucccaca | 80.5 | uugauggccagaaacuucc | ggaagtttctggccatcaa |
| 1153-1177 | (SEQ ID NO: 28) | | (SEQ ID NO: 34) | (SEQ ID NO: 40) |
| GP131 | gcauugauggccagaaacuucccac | 72.1 | auugauggccagaaacuuc | gaagtttctggccatcaat |
| 1154-1178 | (SEQ ID NO: 29) | | (SEQ ID NO: 35) | (SEQ ID NO: 41) |
| GP131 | aaagugaagucaaacugguccugcu | 85.6 | agugaagucaaacuggucc | ggaccagtttgacttcact |
| 1322-1346 | (SEQ ID NO: 30) | | (SEQ ID NO: 36) | (SEQ ID NO: 42) |

### Example 4

### MEF Transformation

This example describes a protocol for using the candidate gene to transform mouse embryonic fibroblasts.

MEFs are isolated from 100 individual 13.5-day-old embryos. The isolated MEFs are pooled and grown in DMEM supplemented with 10% FBS, penicillin and streptomycin. Pooled early-passage (passages 4 to 6) p16^{Ink4a}/p19^{Arf} −/− mouse embryonic fibroblasts are transfected with (1) pKO-Myc; (2) pKO-Myc and pT24-RasV12; (3) pKO-Myc and GP131 cDNA; or (4) pKO-Myc, pT24-RasV12, and GP131 cDNA. All transfections are done in duplicate cultures (8×10⁵ cells) using Lipofectamine Plus in Optimem (Gibco) without serum or antibiotics. Cultures are split 1:3 a day after transfection. Foci are counted on day 12.

### Example 5

### RNAi on Human or Mouse model Cancer Cell Lines in SCIDs

Human cancer cell lines expressing high levels of the candidate, or cell lines established from the inducible mouse cancer model described above, are transfected with a vector encoding a short hairpin RNA homologous to the candidate cancer-related gene. Expression of the RNA is placed under the control of an inducible U6 promoter. Stable cell lines are established. 5×10⁵ cells are injected subcutaneously into the flank of 6 week old female inbred SCID mice. Tumor formation is observed visually. For cell lines derived from the inducible mouse model, tumor formation is induced by doxycycline. Expression of the RNAi is induced once tumors were visually identified. In the case of mouse model-derived tumor cells, the mice are fed with doxycycline. Tumor regression is followed using calipers to measure the shrinking tumor diameter.

### Example 6

### Tumor Formation in SCIDs

Tumor cells derived from the tumor suppressor null (INK4/arf −/−) doxycycline-inducible oncogene mouse model are infected with retrovirus encoding the candidate gene under the control of an IPTG-inducible promoter. Stable cell lines are established. 10⁶ cell are injected subcutaneously into the flank of 6 week old female inbred SCID mice. Mice are fed with doxycycline for 7-12 days. 24 hours prior to doxycycline withdrawal, mice are fed IPTG. IPTG feeding is maintained after withdrawal of doxycycline and tumor regression is monitored using calipers.

### Example 7

### Ceramide Kinase Assay

Ceramide Kinase (CerK) is an enzyme found in the sphingolipid metabolic pathway. CerK catalyzes the transfer of a phosphoryl group from adenosine-5′-triphosphate (ATP) to ceramide to generate ceramide-1-phosphate (C-1-P) and adenosine-5′-diphosphate (ADP).

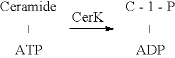

We have developed a mixed micelle coupled enzyme assay system that allows for a rapid CerK assay. The CerK reaction is coupled to a pyruvate kinase/lactate dehydrogenase coupled assay system in a detergent-based mixed micelle format.

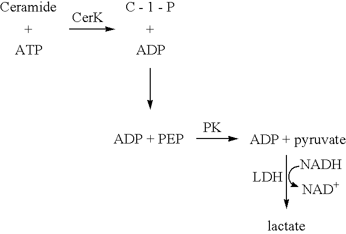

The CerK reaction is indirectly monitored by observing the decrease in NADH by either absorption or fluorescent spectroscopy. This coupled assay system can be used screen to screen for inhibitors for CerK.

The assay final concentrations were 6 mM Triton X-100, 6 mM Triton X-114, 3 mM C16SB (Zwitterionic detergent), 2 mol % ceramide (in mixed micelles) 1 mM DTT, 3 mM CaCl₂, pH 8.0, 0 to 1 mM inhibitor, 5 mM Mg-ATP, 4 mM phosphoenolpyravate (PEP), 0.3 mM NADH, 5 U pyruvate kinase (PK), and lactate dehydrogenase (LDH), and CerK. Ceramide and inhibitor were prepared by combining substrate or inhibitor in Triton mixed micelle system and sonicated to induce micelle formation. Inhibitor (10 μL) was place in well of 96-well plate. A master mix containing all the above components except the enzyme was prepared. The master mix was incubated for 5 min at room temperature. CerK was added and then 90 μL of master mix was added to each well to initiate the reaction. The reaction is monitored using absorption spectroscopy at 340 nm or by fluorescent spectroscopy with excitation at 340/60 nm and emission at 460/40 nm.

What is claimed is:

1. An interfering RNA that inhibits the expression of GP131.

2. The interfering RNA of claim 1, wherein the interfering RNA targets the sequence of SEQ ID NO: 3, SEQ ID NO: 4, or SEQ ID NO: 5.

3. The interfering RNA of claim 1, wherein the interfering RNA inhibits tumorigenesis, tumor development, tumor maintenance, tumor recurrence, tumor growth, or growth of tumor cells in vitro.

4. A method of inducing apoptosis in a cell, comprising contacting the cell with an effective amount of the interfering RNA of claim 1.

5. A method of treating a hyperproliferative condition in a mammal, comprising administering to the mammal an effective amount of the interfering RNA of claim 1.

6. The method of claim 5, wherein the hyperproliferative condition is a cancer.

7. The method of claim 5, further comprising the step of administering a second therapeutic agent to the mammal.

8. The method of claim 6, wherein said second therapeutic agent is selected from the group consisting of an anti-angiogenic agent, anti-metastatic agent, agent that induces hypoxia, agent that induces apoptosis, and an agent that inhibits cell survival signals.

9. An antibody that specifically binds to GP131 and inhibits GP131 activity.

10. The antibody of claim 1, wherein the antibody inhibits tumorigenesis, tumor development, tumor maintenance, tumor recurrence or tumor growth.

11. A method of inducing apoptosis in a cell, comprising contacting the cell with an effective amount of the antibody of claim 9.

12. A method of treating a hyperproliferative condition in a mammal, comprising administering to the mammal an effective amount of the antibody of claim 9.

13. The method of claim 12, wherein the hyperproliferative condition is a cancer.

14. The method of claim 12, further comprising the step of administering a second therapeutic agent to the mammal.

15. The method of claim 14, wherein the second therapeutic agent is selected from the group consisting of an anti-angiogenic agent, anti-metastatic agent, agent that induces hypoxia, agent that induces apoptosis, and an agent that inhibits cell survival signals.

16. A host cell comprising a recombinant DNA comprising a GP131-encoding sequence operably linked to an expression control sequence, wherein the host cell further comprises a genetic mutation that causes the host cell to have a greater likelihood of becoming a cancer cell than a cell not comprising the genetic mutation.

17. The cell of claim 16, where the genetic mutation is in a tumor suppressor gene.

18. A genetically modified non-human mammal at least some of whose cells comprise a genome comprising: (a) a recombinant GP131-encoding nucleic acid operably linked to an expression control sequence, and (b) a genetic mutation that causes the mammal to have a greater susceptibility to cancer than a mammal not comprising the genetic mutation.

19. The genetically modified nonhuman mammal of claim 18, where the genetic mutation is in a tumor suppressor gene.

20. The genetically modified nonhuman mammal of claim 18, wherein the mammal is a transgenic mammal, all of whose cells comprise a recombinant GP131-encoding nucleic acid operably linked to an expression control sequence, and a genetic mutation that causes the mammal to have a greater susceptibility to cancer than a mammal not comprising the genetic mutation.

21. The genetically modified nonhuman mammal of claim 18, wherein the mammal is a chimeric mammal at least some of whose, but not all of whose, somatic cells comprise a recombinant GP131-encoding nucleic acid operably linked to an expression control sequence, and a genetic mutation that causes the mammal to have a greater susceptibility to cancer than a mammal not comprising the genetic mutation.

22. The chimeric mammal of claim 21, wherein the percentage of somatic cells comprising a recombinant GP131-encoding nucleic acid operably linked to an expression control sequence, and a genetic mutation that causes the mammal to have a greater susceptibility to cancer is between 5% and 95%.

23. The chimeric mammal of claim 22, wherein the percentage of somatic cells comprising the recombinant GP131-encoding nucleic acid operably linked to an expression control sequence, and the genetic mutation that causes the mammal to have a greater susceptibility to cancer is between 15% and 85%.

24. The genetically modified nonhuman mammal of claim 18, wherein the GP131-encoding nucleic acid is operably linked to a tissue-specific expression system.

25. A genetically modified nonhuman mammal, wherein the genetic modification reduces or eliminates expression of the mammal's endogenous GP131 genes.

26. The mammal of claim 25, wherein the genetic modification is a knockout of at least one of the mammal's endogenous GP131 alleles.

27. The mammal of claim 25, wherein the genetic modification is addition of an RNAi expression construct targeting GP131 gene expression.

28. The mammal of claim 25, wherein the genetic modification eliminates expression of the mammal's endogenous GP131 genes in a tissue-specific manner.

29. The mammal of claim 25, wherein the mammal is chimeric with respect to the genetic modification.

30. A screening method for identifying a compound useful for treating a hyperproliferative condition, comprising:
a) identifying a biomarker whose level correlates with inhibition of GP131 activity; and
b) detecting a change in the level of the biomarker in the presence of a test compound relative to the level of the biomarker detected in the absence of the test compound.

31. The method of claim 30, wherein said hyperproliferative condition is cancer.

32. A screening method for identifying a compound useful in treatment of a hyperproliferative condition comprising:
(a) providing an inhibitor of GP131 expression or activity;
(b) identifying a negative control biomarker pattern formed by a plurality of biomarkers in a cancer cell wherein the cell is not contacted with the inhibitor of GP131 expression or activity;
(c) identifying a positive control biomarker pattern formed by a plurality of biomarkers in the cancer cell wherein the cancer cell is contacted with the inhibitor of GP131 expression or activity;
(d) identifying a test biomarker pattern formed by a plurality of biomarkers in the cancer cell wherein the cancer cell is contacted with a candidate compound but not contracted with the inhibitor of GP131 expression or activity; and
(e) comparing the negative control biomarker pattern, positive control biomarker pattern and test biomarker pattern,
detecting a greater similarity between the positive control biomarker pattern and the test biomarker pattern than between the negative control biomarker pattern and the test biomarker pattern.

33. A method of claim 30, wherein the said hyperproliferative condition is cancer.

34. A screening method for identifying a compound that inhibits GP115 activity, comprising:
(a) providing a suitable ceramide preparation containing ceramide and ATP,
(b) adding to the preparation a test compound,
(c) adding to the preparation a suitable amount of GP131 (ceramide kinase), and
(d) detecting a decrease in ceramide phosphate production in the presence of the test compound relative to ceramide phosphate production in the absence of the test compound.
